(19) United States
(12) Patent Application Publication
(10) Pub. No.: US 2024/0390144 A1
(43) Pub. Date: Nov. 28, 2024

(54) TETHER FOR DELIVERY OF CARDIAC VALVE

(71) Applicant: Shifamed Holdings, LLC, Campbell, CA (US)

(72) Inventors: Jasper Ellington ADAMEK-BOWERS, San Francisco, CA (US); Jordan SKARO, San Jose, CA (US); Jonathan Hwan OAKDEN, San Jose, CA (US); Uyenchi HA, San Jose, CA (US); Andrew BACKUS, Santa Cruz, CA (US)

(21) Appl. No.: 18/693,856

(22) PCT Filed: Sep. 2, 2022
(86) PCT No.: PCT/US2022/075931

§ 371 Date: Mar. 20, 2024

Related U.S. Application Data
(60) Provisional application No. 63/261,498, filed on Sep. 22, 2021.

(51) Int. Cl. A61F 2/24

(52) U.S. Cl. CPC A61F 2/2457; A61F 2/2466; A61F 2220/0008; A61F 2230/0091; A61F 2250/0018

(57) ABSTRACT

Devices and systems for treatment of a diseased heart valve. The anchor may be deployed around chordae and/or leaflets of the diseased valve prior to delivery of a valve prosthesis into the annulus of the diseased valve and within the anchor. A tether may be configured to provide access to the anchor and/or adjust a position of the anchor with respect to the diseased valve. The tether may include a proximal region and a distal region, where the distal region may be releasably connectable to the anchor. The proximal and distal regions may have different properties, such as different flexibilities. In some cases, at least a portion of the tether may be configured to transition between a flexible and stiff state.

### CROSS REFERENCE TO RELATED APPLICATIONS

This application claims priority to U.S. Provisional Application No. 63/261,498, filed on Sep. 22, 2021, entitled “TETHER FOR DELIVERY OF CARDIAC VALVE”, the entirety of which is incorporated herein by reference for all purposes.

### INCORPORATION BY REFERENCE

All publications and patent applications mentioned in this specification are herein incorporated by reference to the same extent as if each individual publication or patent application was specifically and individually indicated to be incorporated by reference.

### FIELD

Medical devices and systems for use in the delivery of prosthetic cardiac valves. In some examples, the devices and systems are used in delivering prosthetic mitral valves.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transeptally, requiring transapical access instead.

Thus, a new valve delivery system or method that overcomes some or all of these deficiencies is desired.

### SUMMARY

Described herein are devices, systems and methods for use in delivery of prosthetic heart valves, such as prosthetic mitral valves. The devices, systems and methods may allow for less invasive and quicker surgical procedures. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

According to some aspects, a system for treating a diseased native valve of a heart comprises: an anchor having a spiral shape that is configured to engage with chordae tendineae and/or leaflets of the diseased native valve and to provide a securing force around a valve prosthesis; and a tether configured to provide access to the anchor when the anchor is deployed around the chordae tendineae and/or leaflets of the native valve, the tether having a distal region and a proximal region, the distal region having a distal end connected to the anchor, wherein the distal region and the proximal region have different bending stiffnesses.

The tether may have a sufficiently high tensile strength to support an anchor delivery catheter and a valve delivery catheter tracked thereover. The distal region may have a sufficiently high tensile strength to allow a user to manipulate a position of the anchor when the anchor is deployed around the chordae of the native valve. The proximal region may have a sufficiently high compressive strength to resist axial compression when an axial compressive force is applied to the tether, and a sufficiently high tensile strength to resist axial elongation when an axial tensile force is applied to the tether. The distal end of the tether may be releasably connected to the anchor. The distal region may have a lower bending stiffness than the proximal region.

The tether may include an inner cable housed within a tubular housing. The cable may be translatable within the tubular housing. The tubular housing may include one or more of: a tubular coil, a braided tube, and a tubular jacket. The tubular coil at the distal region of the tether may have a larger pitch than the tubular coil at the proximal region of the tether. The braided tube at the distal region of the tether may have a higher pics per inch (PPI) braid than the braided tube at the proximal region of the tether. The tubular jacket at the distal region of the tether may be made of a low durometer material than the tubular jacket at the proximal region of the tether. The tubular housing may include one or more radio-opaque markers. The tubular housing at the proximal region may include a stacked tubular coil, and the tubular housing at the distal region may include a slitted tube.

The distal region (of the tether) may be configured to transition between a flexible state and a stiff state, wherein the distal region may have a pre-determined shape when in the stiff state. The pre-determined shape may include an L-shaped bend. The pre-determined shape may include a U-shaped bend. The distal region may have a shorter length than the proximal region. A length of the distal region may range between 5% and 20% of a length of the proximal region. A diameter of the distal region may range between 0.02 inches and 0.08 inches. The distal region may have an axial length that is at least double an axial length of the valve prosthesis.

According to some aspects a method of treating a diseased native valve of a heart comprises: advancing an anchor delivery catheter toward the diseased native valve, the anchor delivery catheter including an anchor and a tether housed therein, the tether including a distal region and a proximal region, wherein the distal region is connected to the anchor and is more flexible than the proximal region; deploying the anchor from the anchor delivery catheter, wherein the anchor encircles chordae and/or leaflets of the diseased native valve, and the distal region of the tether extends from the anchor delivery catheter to the anchor; tracking a valve delivery device over the distal region of the tether; and releasing a valve prosthesis from the valve delivery device into the annulus of the diseased native valve and within the anchor. Deploying the anchor may comprise pushing the tether and the anchor through a curved anchor guide.

The method may further comprise, after deploying the anchor, translating a portion of the distal region of the tether through a central opening of the anchor such that the portion of the distal region of the tether assumes an inverted configuration inferior to the deployed anchor. The portion of the distal region may include a U-shaped bend when in the inverted configuration. The distal region has a shorter length than the proximal region. A length of the distal region may range between 5% and 20% of a length of the proximal region. A diameter of the distal region may range between 0.02 inches and 0.08 inches. The distal region may have a length that is at least double an axial length of the valve prosthesis.

According to some aspects a system for treating a diseased native valve of a heart comprises: an anchor shaped and sized to provide a securing force around a valve prosthesis, the anchor having a spiral shape that is configured to engage with chordae and/or leaflets of the diseased native valve; and a tether attached to the anchor and configured to adjust a position of the anchor with respect to the native valve, wherein at least a portion of the tether is configured to transition between a flexible state and a stiff state.

The at least a portion of the tether may have a pre-determined shape when in the stiff state. The pre-determined shape may include at least one bend.

The tether may include a distal region and a proximal region, wherein a distal end of the distal region is attached to the anchor, wherein the distal region is configured to transition between the flexible state and the stiff state. A lateral flexibility of the distal region in the flexible state may be greater than a lateral flexibility of the proximal region. The distal region may have a shorter length than the proximal region. A length of the distal region may range between 5% and 20% of a length of the proximal region. A diameter of the distal region may range between 0.02 inches and 0.08 inches. The distal region may have a length that is at least double an axial length of the valve prosthesis.

The anchor may be sized and shaped to extend around a frame structure of the valve prosthesis.

The system may further comprise an actuator at a proximal end of the tether, the actuator configured to control transition the tether between the flexible and stiff states. The actuator may further be configured to control engagement of the tether with the anchor.

The tether may be configured to releasable lock with the anchor, wherein the anchor is prevented from rotating relative to the tether when locked together.

The tether may comprise an inner cable housed within a tubular housing. The tubular housing may be configured to bend and stiffen to a predetermined shape upon tensioning of the inner cable. The predetermined shape may include an L-shaped bend. The predetermined shape may include a U-shaped bend. Translating the inner cable within the tubular housing may cause tensioning of the inner cable. The tubular housing may include a tubular component having a side with a plurality of transverse slits, wherein the slits are arranged to cause the component to preferentially bend in a direction toward the side of the component with the plurality of slits. The tubular housing may include a sheath covering the component.

The tether may be releasably attached to the anchor.

The system may further comprise an outer sheath as part of an anchor delivery catheter system, wherein the tether is configured to translate within the outer sheath.

The system may further comprise an anchor guide that is configured to translate within the outer sheath, wherein the tether is configured to translate within the anchor guide.

According to some aspects a method of treating a diseased native valve of a heart comprises: advancing an anchor delivery catheter toward the diseased native valve, the anchor delivery catheter including an anchor and a tether housed therein, the tether configured to transition between a flexible state and a stiff state, wherein the tether is in the flexible state when housed within the anchor delivery catheter; deploying the anchor from the anchor delivery catheter such that the anchor encircles chordae and/or leaflets of the diseased native valve, wherein the tether is connected to the deployed anchor; and transitioning the tether to the stiff state to cause the tether to bend, wherein bending the tether adjusts a position of the deployed anchor.

The method may further comprise releasing a valve prosthesis from a valve delivery device into the annulus of the diseased native valve and within the deployed anchor.

The method may further comprise disconnecting the tether from the deployed anchor.

The tether may include a cable housed within a tubular housing, wherein transitioning the tether to the stiff state comprises applying tension on the cable. Applying tension on the cable may comprise manipulating an actuator. Applying tension on the cable may comprise pulling the cable proximally. Adjusting the position of the deployed anchor may comprise moving the deployed anchor toward an annulus of the diseased valve. Adjusting the position of the deployed anchor may comprise orienting a plane of the deployed anchor in alignment with an annulus of the diseased valve.

The tether may bend to form an L-shaped bend.

The method may further comprise translating the tether through an opening of the deployed anchor and positioning the tether in an inverted configuration.

The tether may bend to form a U-shaped bend.

According to some aspects a method of treating a diseased native valve of a heart comprises: advancing an anchor delivery catheter toward the diseased native valve, the anchor delivery catheter including an anchor and a tether housed therein, the tether configured to transition between a flexible state and a stiff state, wherein the tether is in the flexible state when housed within the anchor delivery catheter; extending the anchor and a distal region of the tether from the anchor delivery catheter within the heart; and transitioning the tether to the stiff state to cause the tether to bend, wherein bending the tether steers the anchor such that the anchor is deployed around chordae and/or leaflets of the diseased native valve.

These and other aspects and features are disclosed herein.

All of the methods and apparatuses described herein, in any combination, are herein contemplated and can be used to achieve the benefits as described herein.

### BRIEF DESCRIPTION OF THE DRAWINGS

Novel features described herein are set forth with particularity in the appended claims. A better understanding of the features and advantages of the embodiments may be obtained by reference to the following detailed description that sets forth illustrative embodiments and the accompanying drawings.

FIGS. 1A-1G illustrate sequential views of an exemplary method of implanting an anchor for a valve prosthesis near a native valve annulus.

FIGS. 2A-2K illustrate sequential views of an exemplary method of delivering a valve prosthesis into an anchor previously placed near a native valve annulus.

FIGS. 3A-3H illustrate sequential views of an exemplary method of inverting a tether extending from an anchor implanted near a native valve annulus.

FIG. 4A illustrates a diagram of an exemplary tether system for implanting an anchor and/or valve prosthesis.

FIG. 4B illustrates an exemplary interface between a tether and an anchor.

FIG. 4C illustrates an exemplary connector for connecting a tether to an anchor.

FIG. 4D illustrates an exemplary tether having a proximal region and a distal region.

FIGS. 5A-5C illustrate examples of different types of tethers having a flexible distal region.

FIGS. 6A-6E illustrate an exemplary tether housing having a flexible distal region.

FIG. 7 illustrates another exemplary tether housing that is a variation of the tether housing of FIGS. 6A-6E.

FIGS. 8A-8C illustrate another exemplary tether housing that is a variation of the tether housings of FIGS. 6A-6E and FIG. 7.

FIG. 9 is a flowchart illustrating an exemplary method of using a tether having a flexible distal region.

FIG. 10A illustrates an exemplary component of a tether housing that provides variable flexibility.

FIG. 10B illustrates another exemplary component of a tether housing that provides variable flexibility.

FIGS. 11A-11C illustrate an exemplary variably flexible tether at different degrees of bending.

FIGS. 12A and 12B illustrate an exemplary actuator for a variable flexible tether.

FIGS. 13A-13C illustrate another exemplary actuator for a variable flexible tether.

FIGS. 14A and 14B illustrate a further exemplary actuator for a variable flexible tether.

FIGS. 15A and 15B illustrate another exemplary variably flexible tether in a flexible state and a stiff state.

FIG. 16 is a flowchart illustrating an exemplary method of using a variably flexible tether.

FIG. 17 is a flowchart illustrating another exemplary method of using a variably flexible tether.

### DETAILED DESCRIPTION

Described herein are devices and methods for use in delivering a prosthetic cardiac valve system, for example during a mitral valve replacement. The prosthetic valve system can include a prosthetic valve, which is configured to replace a diseased native valve, and an anchor, which is configured to secure the prosthetic valve in place within the annulus of the diseased native valve. In some cases, the anchor and the prosthetic valve are delivered to the heart separately. In some cases, the anchor is delivered to the heart using an anchor delivery catheter system and the prosthetic valve is delivered to the heart using a valve delivery catheter system. In other cases, the anchor and prosthetic valve are delivered to the heart using a single delivery catheter system.

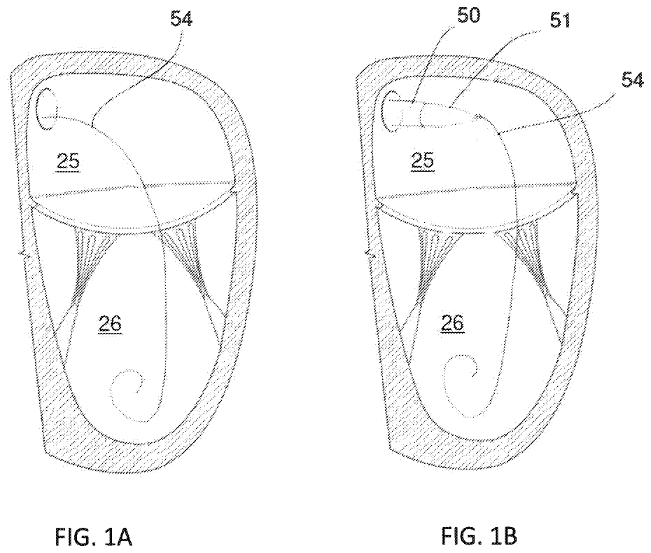

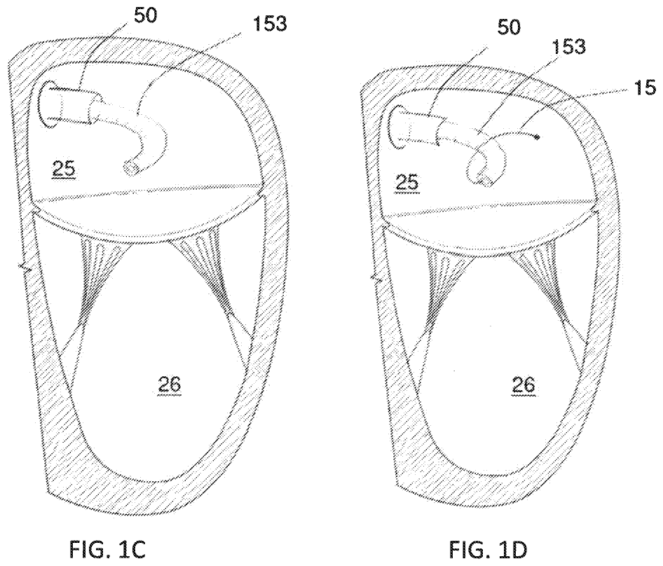

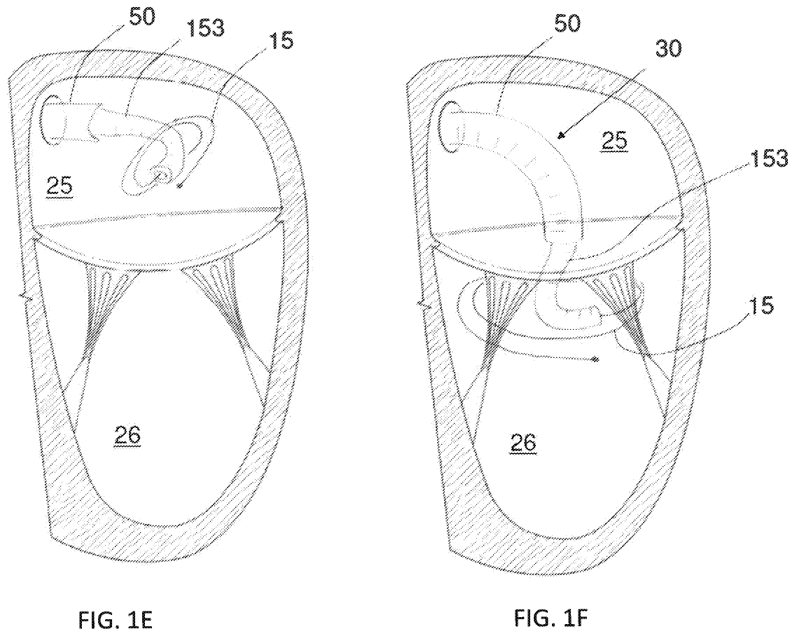

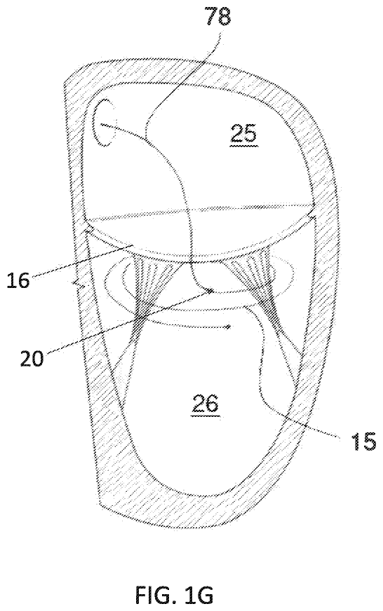

The devices and method described herein relate to a tether that is attached to the anchor and that is configured to extend out of the heart, and in some cases, out of the patient's body. In some examples, the tether may be used to guide positioning of the anchor and/or placement of the valve prosthesis within the native valve. The tethers described herein can include one or more features designed to enhance the capability of the tether for positioning the anchor and/or valve prosthesis. In some cases, the tether includes regions having different degrees of flexibility/bendability and/or shapes. FIGS. 1A-1G, 2A-2K and 3A-3H are presented to illustrate examples of how tethers may be used in the delivery of an anchor and a prosthetic valve.

FIGS. 1A-1G show sequential views of a method of implanting an anchor 15 for a valve prosthesis using an anchor delivery system. At FIG. 1A, a transseptal puncture is made. A guidewire 54 is then routed through the puncture site and left either in the left atrium 25 or across the mitral valve into the left ventricle 26. At FIG. 1B, the outer sheath 50 (also referred to as an anchor delivery catheter) of the anchor delivery system is tracked over the guidewire 54 until the distal end of the outer sheath 50 protrudes into the left atrium 25. In some examples, the outer sheath optionally includes an inner dilator 51. The guidewire 54 and inner dilator 51 (if used) are then removed from the outer sheath 50. At FIG. 1C, the inner sheath and anchor guide 153 are inserted through the outer sheath 50 until the distal tip of the anchor guide 153 extends into the left atrium 25. The anchor guide 112 can be configured to take on a pre-determined curved shape. The anchor guide 153 can be positioned and/or oriented as desired by steering the distal end of the sheath 50 and/or rotating the inner shaft and anchor guide 153 within the sheath 50. In some examples, the distal portion of the outer sheath 108 is bendable between a straight configuration and a bent configuration. Such bending can be controlled, for example, at a handle operationally connected to the anchor delivery catheter. At FIG. 1D, once the anchor guide 153 is in the correct orientation, the anchor 15 can be pushed out through distal tip of the anchor guide 153. The anchor 15 can be, or can include, a wire that takes on a particular shape. In this example, the anchor 15 can be configured to take on a spiral shape with the wire circling around central axis and extending around the chordae tendineae when implanted. At FIG. 1E, the curvature of the anchor guide 153 can cause torsion on the anchor 15, causing the anchor 15 to deploy concentrically with the outer sheath 50 into the atrium 25. At FIG. 1F, the entire delivery system can be pushed and steered (for example, via steering mechanisms in the outer sheath 50) towards an apex of the ventricle 26, crossing through the mitral valve. In some embodiments, counter-rotation of the anchor 15 (via counter-rotation of the inner shaft and guide 153) may aid in getting the anchor across the mitral valve without tangling. Once the anchor 15 is at the correct depth within the ventricle 26, forward rotation of the anchor 15 (via forward rotation of the inner shaft and guide 153) will allow the anchor 15 to encircle the mitral leaflets and chordae. In some embodiments, the anchor 15 can be deployed towards the apex to avoid interference with mitral leaflet motion. At FIG. 1G, the outer sheath 40, inner sheath, and anchor guide 153 are removed, leaving a tether 78 in place (and still attached to the anchor 15). A frame structure of the valve prosthesis can then be delivered over the tether 78 and into place within the mitral valve using a valve delivery system.

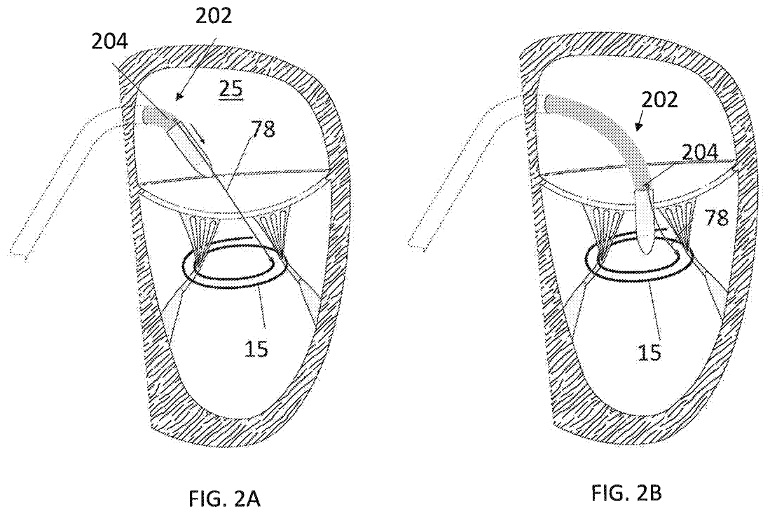

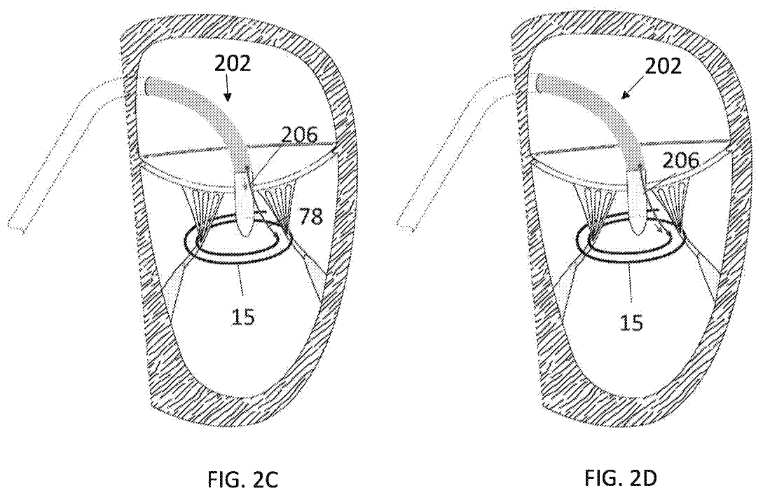

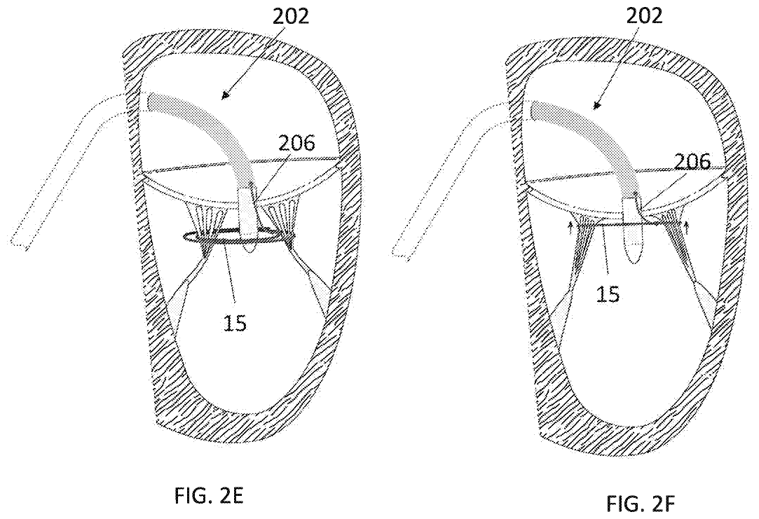

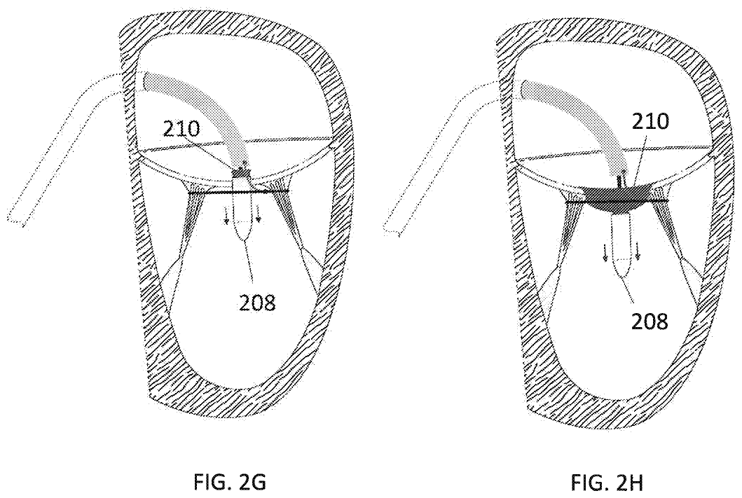

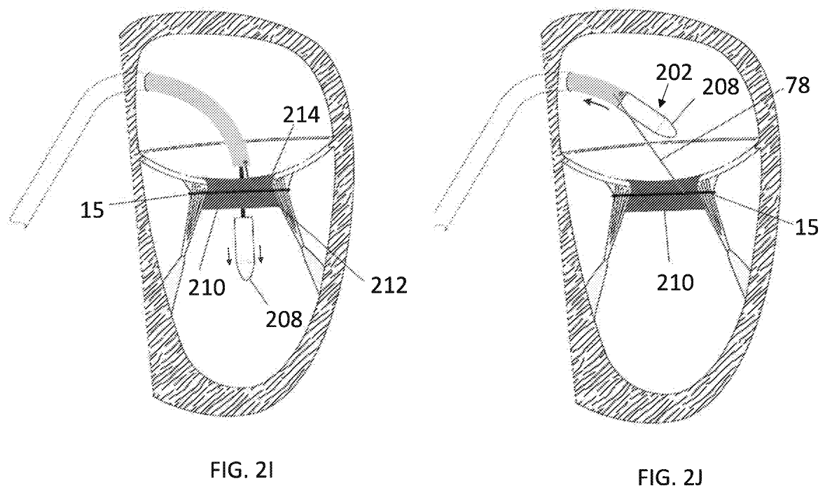

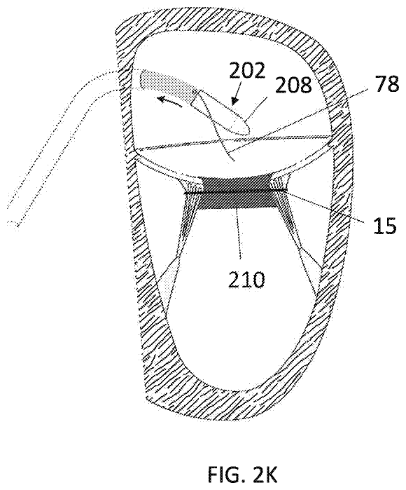

FIGS. 2A-2K show an example method of delivering a prosthetic valve using a valve delivery system to an anchor having a tether, after the anchor has already been placed (e.g., as shown in FIG. 1G). A proximal portion (not shown) of the tether 78, that is external to the patient, can be loaded into a valve delivery system 202 (e.g., via port 204) to enable the valve delivery system 202 to be tracked thereover and into the left atrium 25 (e.g., as shown in FIG. 2A). The tether 78 can guide and/or promote a positional relationship between anchor 15 and valve delivery system 202. As shown in FIG. 2B, the valve delivery system 202 is pushed across the mitral valve. In this example, the tether 78 can be positioned within a (e.g., separate) lumen within the valve delivery catheter and exit through a side port 204, as shown in FIGS. 2A and 2B. Other embodiments for carrying the tether 78 are also possible. For example, the valve delivery catheter may include a monorail for carrying the tether 78. In other cases, the tether 78 may be carried within an inner shaft of the valve delivery catheter.

FIG. 2C shows a positioning tool 206 being tracked over the tether 78. Referring to FIG. 2D, the positioning tool 206 is advanced distally over the tether 78 until a distal end of the positioning tool 206 meets a proximal end of the anchor 15. The positioning tool 206 can be positioned or steered in a way to transition the positioning tool 206 from a first (e.g., flexible) configuration to a second (e.g., stiffened) configuration. In the second configuration, the positioning tool 206 can be used to maintain or adjust a position the anchor 15. For example, the positioning tool 206 can be used to achieve axial alignment between the anchor 15 and the valve delivery system and perpendicular alignment to the native valve. The positioning tool 206 can also be used to pull the anchor up, as shown in FIGS. 2E and 2F, towards mitral annulus to better engage with mitral leaflets and reduce leakage around valve/anchor (paravalvular leakage). FIG. 2F shows the anchor in position near the mitral annulus. The positioning tool 206 can be used to position the anchor 15 in a way that achieves planarity with the mitral annulus, which can help ensure planar positioning of the prosthetic valve frame with respect to the mitral annulus. The positioning tool 206 can also ensure that the anchor 15 maintains good encircling of the native anatomy (e.g., chordae and leaflets).

Referring to FIG. 2G, once the anchor 15 is properly positioned, a valve capsule 208 can be translated distally, exposing the prosthetic valve frame 210. FIG. 2H shows the valve capsule 208 continuing to move distally, exposing more of the prosthetic valve frame 210. As more of the prosthetic valve frame 210 is exposed, the exposed portions may begin to expand, as depicted in FIG. 2H. A deployment mechanism can maintain an axial height relationship between the prosthetic valve 210, anchor 15, and the native valve anatomy. For example, a steering mechanism of the valve sheath of the valve delivery system can be used to control directional position.

Referring to FIG. 2I, the valve capsule 208 continues to translate distally, until the entire valve frame 210 is exposed. The valve frame 210 is held in place using a compression fit between the valve frame 210 and the anchor 15. As shown in FIGS. 2H and 2I, the valve frame 210 may comprise a ventricular flare 212 and an atrial flare 214 with a waist positioned around a midsection of the valve frame 210. The valve frame 210 may seat against the anchor 15 such that the anchor 15 surrounds the waist section of the valve frame 210. FIG. 2J shows the valve delivery system 202 being retracted and removed from the center of the valve frame 210. FIG. 2K shows the tether 78 detached from the anchor 15 and partially retracted into the valve delivery system 202. The valve delivery system 202 (including the valve capsule 208, positioning tool 206 and the tether 78) can then be withdrawn and removed from the body, leaving the anchor 15 and prosthetic valve 210 in place.

In some examples, the tether 78 may be positioned in an inverted configuration prior to tracking of the valve delivery system 202 over the tether 78. In the inverted configuration, a portion the tether 78 is positioned through the deployed anchor 15 and within the left ventricle 26. This inverted configuration may advantageously position the tether 78 inferior to the deployed anchor 15 and for adjusting the deployed anchor in a direction toward the atrium and the native valve plane. The positioning tool (if used) tracked over the inverted tether 78 may then be used to adjust a position of the anchor 15 closer toward the native valve plane and in alignment with a midsection of the prosthetic valve 210.

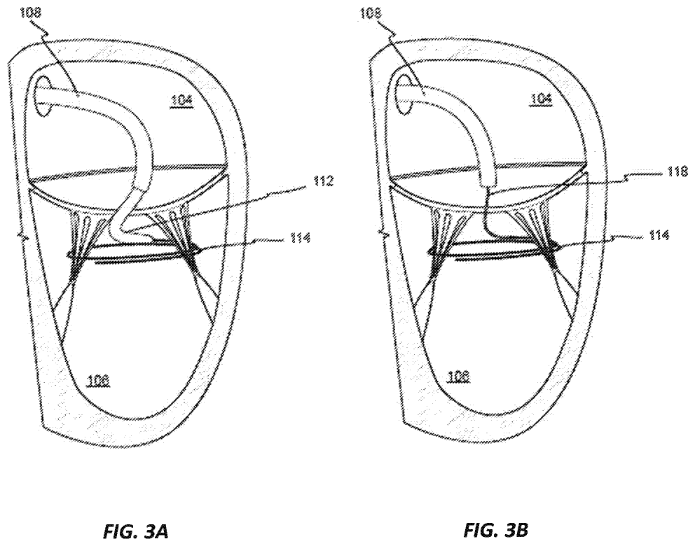

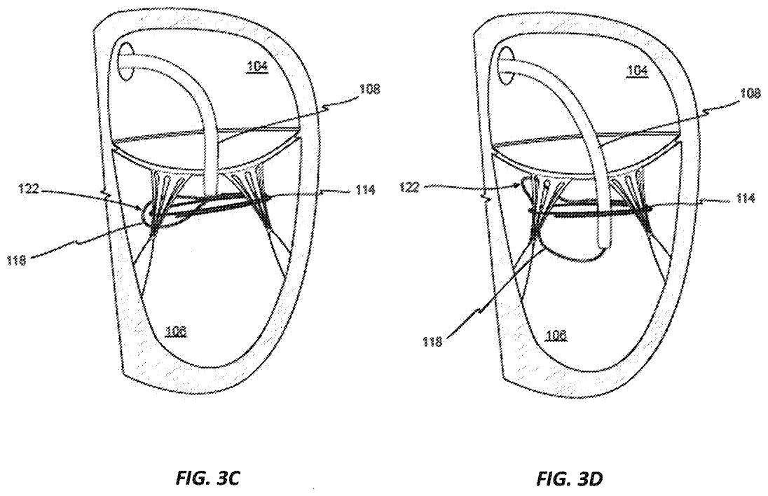

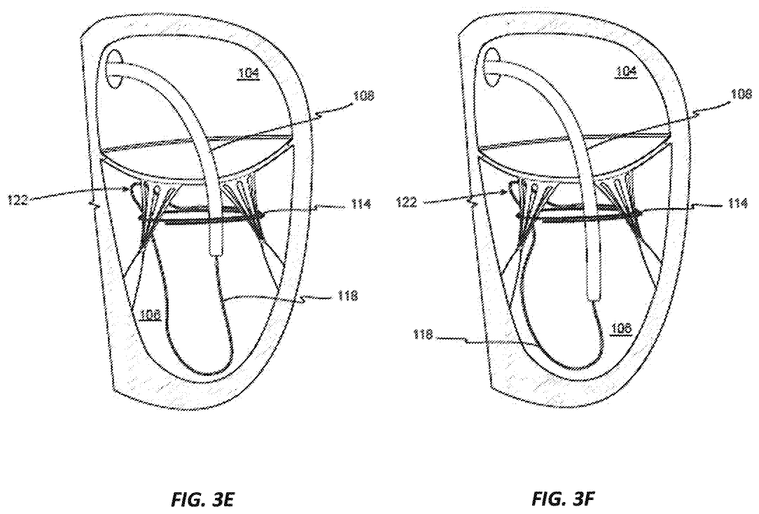

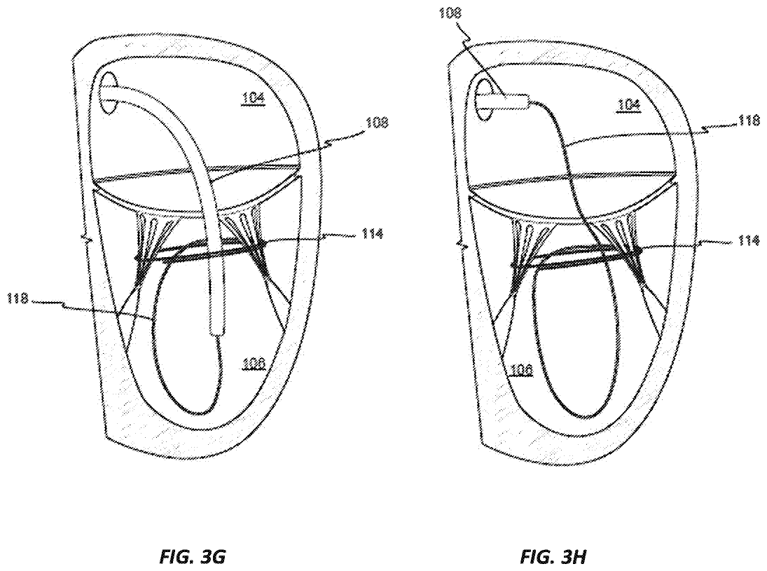

FIGS. 3A-3H show an example method of inverting a tether 118, according to some embodiments, as part of delivering an anchor of a prosthetic valve system within a patient. FIG. 3A shows an anchor guide 112 after being at least partially pushed across the mitral valve plane and used to position the anchor 114 such that the anchor 114 encircles the mitral leaflets and/or chordae (like as shown in FIG. 1G). At FIG. 3B, the distal end of the anchor guide 112 is retracted proximally into the outer sheath 108 and the tether 118 emerges from the distal end of the outer sheath 108. The tether 118 can be configured to be sufficiently flexible to bend laterally as it traverses the patient's vessels and heart, but also be sufficiently stiff to resist becoming tangled as it is manipulated once out of the outer sheath 108. The stiffness of the tether 118 may provide some resistance when attempting to feed the tether 118 through the anchor 114.

A steerable distal tip of the outer sheath 108 by be used to advance the tether 118 through the anchor 114. For example, at FIG. 3C, the outer sheath 108 is advanced through the native valve annulus (e.g., coaxially) using the steering mechanism of the outer sheath 108. In addition, the tether 118 can be fed distally through the outer sheath 108 to provide a slack 122 of tether 118 to coil within the atrium 104. At FIG. 3D, the distal end of the outer sheath 108 is advanced further through the central opening of the annular-shaped anchor 114, and across the plane of the anchor 114, to allow a loop of the tether 118 to enter the left ventricle 106. At FIG. 3E, the tether 118 is further fed through the outer sheath 108 and deployed into the left ventricle 106. At FIG. 3F, the outer sheath 108 is advanced further toward the left ventricle apex. In some cases, the tether 118 may become constrained within the anchor 114 due to tension being put the tether 118 as it is advanced through the annulus of the anchor 114. At FIG. 3H, the tether 118 is retracted proximally with respect to the outer sheath 108 enough to remove at least a portion of the slack 122 of the tether 114 remaining within the left atrium 104, while still leaving enough of the length of the tether 118 within the ventricle 106 to enable access to the anchor 114 from a sub-annular position with respect to the anchor 114 during delivery of the prosthetic valve. As shown, this can allow the tether 118 to unwind from a constrained/coiled configuration to an inverted configuration, where the tether 118 reverses its orientation and the sub-annular portion of the tether 118 takes on a U-shaped bend. At FIG. 3I, the outer sheath 108 is retracted proximally leaving the tether 118 in the inverted position within the left ventricle 106 for subsequent concentric delivery of the prosthetic valve over the tether 118.

The anchor and valve deployment methods shown in FIGS. 1A-1G, FIGS. 2A-2K and FIGS. 3A-3H are presented only as examples. Any of a number of anchor deployment and valve deployment methods may be used in accordance with the devices and methods described herein. Example valve prostheses and related aspects are described in International Application No. PCT/US2020/027744, filed on Apr. 10, 2020, published as WO 2020/210685, and entitled “MINIMAL FRAME PROSTHETIC CARDIAC VALVE DELIVERY DEVICES, SYSTEMS, AND METHODS,” which is incorporated by reference herein in its entirety for all purposes. Example anchors and related aspects are described in U.S. application Ser. No. 16/723,537, filed on Dec. 10, 2019, published as U.S. Patent Application publication No. U.S. Pat. No. 20,200,261,220A1, and entitled “PROSTHETIC CARDIAC VALVE DEVICES, SYSTEMS, AND METHODS,” which is incorporated by reference herein in its entirety for all purposes. Examples of tether inversion procedures and devices are described in International Application number PCT/US2021/040623, filed on Jul. 7, 2021, and entitled “VALVE DELIVERY SYSTEM,” which is incorporated by reference herein in its entirety for all purposes.

The tethers described herein can include features to enhance the capability and performance of the tether during an anchor deployment process (e.g., FIGS. 1A-1G) and/or a valve prosthesis deployment process (e.g., FIGS. 2A-2K). In some cases, the tethers described herein can be used to enhance the capability and performance of the tether in an inverted configuration (e.g., FIGS. 3A-3H). For example, the tethers can include characteristics that enable the tether to function without the use of a separate positioning tool and/or anchor guide.

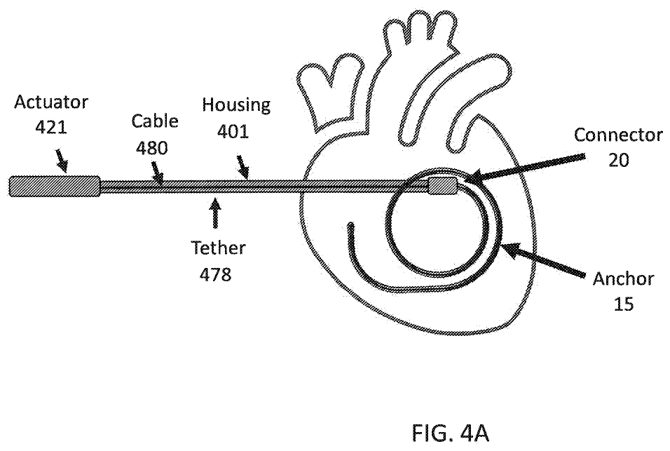

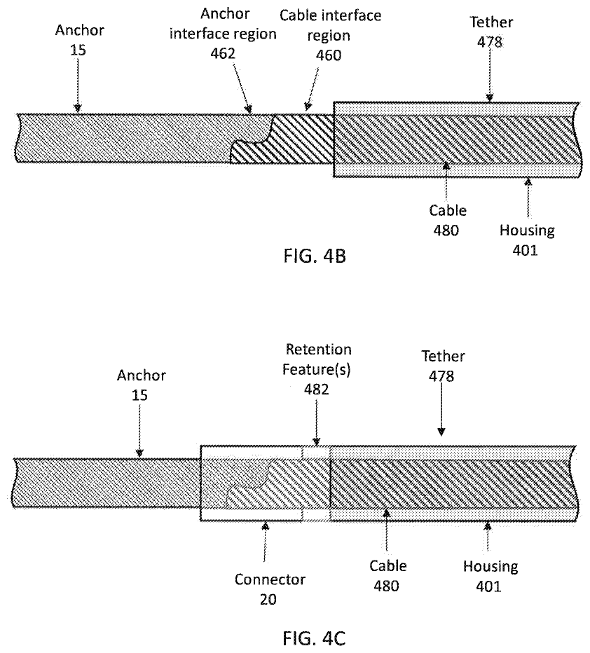

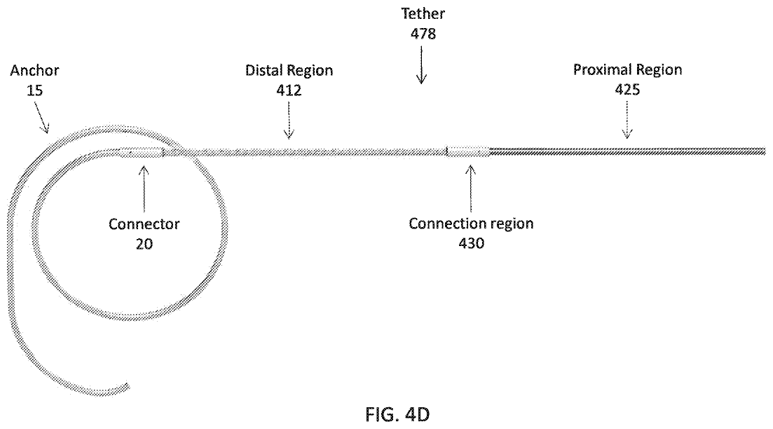

FIG. 4A illustrates a high-level diagram of an exemplary tether system. A tether 478 can be connected to an anchor 15 to provide access to the anchor 15 when the anchor 15 is deployed within the patient heart (e.g., around the chordae tendineae of the native valve). The tether 478 can have a sufficiently high tensile strength to support one or more delivery catheters (e.g., anchor delivery catheter and/or valve delivery catheter) that will be tracked over the tether 478. A distal end of the tether 478 may be coupled to a proximal end of the anchor 15 via a connector 20. The anchor 15 can be configured to transition between an elongate (e.g., straight) shape and a spiral shape as shown in FIG. 4A. The anchor 15 can take on the elongate shape when housed within the anchor delivery catheter (e.g., FIGS. 1A-1C), and take on the spiral shape when deployed from the anchor delivery catheter (e.g., FIGS. 1D-1E). The connector 20 may be configured to secure the anchor 15 to the tether 478 and release the anchor 15 at a prescribed time so that the anchor 15 may be freed from the tether 478 when the tether 78 is no longer needed. For example, the connector 20 can be in a locked state to keep the anchor 15 connected to the tether 478 across procedural loading conditions, such as during delivery of the anchor 15 to the native valve and/or delivery of the prosthetic valve over the tether 478. The connector 20 can be configured to transition to an unlocked state such that the anchor 15 can be freed from the tether 478. In some cases, an actuator 421 controls whether the connector 20 is in a locked or an unlocked state.

The tether 478 can include a cable 480 (also referred to herein as a wire) housed within a tubular housing 401. The cable may be translatable with respect to the housing 401. In some cases, a distal end of the cable may engage with the anchor 15 when the connector 20 is in the locked state, thereby releasably locking the anchor 15 and tether 478 together. When connected in the locked state, the anchor 15 may be prevented from rotating relative to the tether 478. The tubular housing 401 may have enough tensile strength to resist axial compression and/or elongation as pulling and/or pushing forces are applied to the tubular housing 401 when manipulating the anchor 15. The tether 478 may have a small enough diameter for entry into the patient's vessels and/or heart yet be resistant to kinking as it traverses through the patient's vessels and/or heart. In some examples, a diameter of at least a distal region of the tether 478, including the connector 20, may range between any two of the following values: 0.03 inches, 0.04 inches, 0.05 inches, 0.06 inches, and 0.07 inches. For example, the diameter of at least a distal region of the tether 478 may range from 0.03 inches and 0.07 inches.

One or more parts of the tether 478 (e.g., the tubular housing 401 and/or the cable housed therein) may be made of any of a number of materials. In some examples, one or more parts of the tubular housing 401 may comprise one or more polymers, such as a block copolymer comprising polyamide and polyether (e.g., Pebax®). In some examples, one or more parts of the cable housed within the tubular housing 401 may comprise one or more of the following materials: stainless steel, nickel titanium alloy (e.g., nitinol), cobalt chromium nickel alloy (e.g., Elgiloy®), and cobalt chromium

FIG. 4B shows an exemplary interface between an anchor 15 and a tether 478. The tether 478 can include a cable 480 housed within a lumen of a tubular housing 401 (shown in partially transparent view). A cable interface region 460 at a distal end of the cable 480 can be configured to (e.g., directly or indirectly) engage with an anchor interface region 462 at a proximal end of the anchor 15. In some cases, the cable interface region 460 can be part of a member that is coupled (e.g., fixedly) to a remainder of the cable 480 housed within the housing 401. In some cases, the anchor interface region 462 can be part of a member that is coupled (e.g., fixedly) to a remainder of the anchor 15. In some cases, the cable interface region 460 and anchor interface region 462 can have corresponding mating surfaces.

FIG. 4C shows an exemplary connector 20 (shown in partially transparent view) that is configured to releasably connect the tether 478 with the anchor 15. The connector 20 can be configured to maintain connection between the tether 478 and the anchor 15 in a locked state, and to allow the tether 478 to be decoupled from the anchor in an unlocked state. The connector 20 can at least partially surround the cable interface region 460 of the tether 478 and the anchor interface region 462 of the anchor 15. For example, the connector 20 can include a lumen that is configured to accept at least a portion of the cable 480 and the anchor 15. The connector 20 can include one or more retention features 482 that can be configured to prevent the cable 480 and anchor 15 from translating axially (e.g., distally and/or proximally) with respect to the connector 20 when in a locked state. The retention feature(s) 482 can be configured to allow the cable 480 and/or the anchor 15 to translate axially (e.g., distally and/or proximally) with respect to the connector 20 when in the unlocked state. The retention feature(s) 482 may include one or more ledges or shelves within the lumen of the connector 20 that can engage with corresponding engagements feature(s) of the cable 480 and/or anchor 15 when locked within the connector 20. In some cases, the retention feature(s) 482 can be configured to prevent the cable 480 and/or the anchor 15 from rotating with respect to the connector 20 when in the locked state. When locked within the connector 20, the mating surfaces of the interface region 460 and the anchor interface region 462 can prevent the anchor 15 from rotating independently with respect to the cable 480.

FIG. 4D shows a tether 478 illustrating different regions of the tether 478, according to some embodiments. The tether 478 can include a distal region 412 and a proximal region 425. The distal region 412 may be coupled to the proximal region 425 at a connection region 430. In some cases, the connection region 430 may include a connecting member (e.g., joint or adapter), such as shown in the example of FIG. 4D. In other cases, the distal region 412 may be integrally connected to the proximal region 425 at the connection region 430 (e.g., without a distinct connecting member). The distal region 412 can include a distal end that can be releasably attached to the anchor 15 via the connector 20.

The proximal region 425 may be sufficiently stiff for the user to have control over the positioning of the anchor 15. The proximal region 425 may have a sufficiently high column strength to track the anchor through the anchor delivery catheter. The proximal region 425 may be sufficiently stiff to undergo minimal elongation and/or compression under axial load during various procedures, such as during sheathing of the anchor 15.

The tether 478, including the distal region 412 and the proximal region 425, may be resistant to kinking as it traverses through the blood vessels and heart. For example, the proximal region 425 of the tether 478, e.g., the distal end of the tether 478 where the user interacts with the tether 478 (e.g., at actuator 421, FIG. 4A), may be configured to resist kinking when subject to forces that would otherwise cause kinking. For example, the proximal region 425 may have a large enough diameter and/or be made of stiff enough material(s) to resist bending/twisting. The distal region 412 of the tether 478 can advantageously have a large enough diameter and/or be made of sufficiently flexible material(s) to provide kink resistance, yet have a small enough diameter to sufficiently bend within the confines of the heart chamber(s), fit within the anchor delivery catheter and/or interact with various portions of the valve delivery system.

In some examples, the distal region 412 may have a larger outer diameter than the proximal region 425. In other examples, the proximal region 425 may have a larger outer diameter than the distal region 412 (e.g., to provide more column strength and stiffness to the proximal region 425 while maximizing flexibility of the distal region 412).

The distal region 412 (or a portion thereof) can be configured to enter the subject's heart and interact with various components of the anchor delivery system and/or the valve delivery system. The proximal region 425 can be connected to and provide access to the distal region 412, and may include a portion that traverses outside of the subject's body. The distal region 412 may have at least a length that is sufficiently long to allow the prosthetic valve to travel over the distal region 412 at various stages of deploying the prosthetic valve while maintaining control over the prosthetic valve. This length may be referred to as an exchange length. In some cases, the distal region 412 has a length that is at least double an axial length of the prosthetic valve. In some examples, the distal region 412 is shorter than the proximal region 425. In some examples, the distal region 412 can have a length that ranges between any two of the following values: 1 inch, 2 inches, 3 inches, 4 inches, 6 inches, 8 inches, 10 inches, 12 inches, 20 inches and 30 inches. In some examples, the proximal region 325 can have a length that ranges between any two of the following values: 30 inches, 40 inches, 50 inches, 60 inches, 70 inches, 80 inches, 90 inches and 100 inches. In some cases, a combined length of the distal region 212 and the proximal region 425 ranges between any two of the following values: 30 inches, 50 inches, 80 inches, 100 inches, 150 inches and 200 inches. In some cases, a percentage of the length of the distal region 412 compared to the length of the proximal region 425 ranges between any two of the following values: 1%, 5%, 10%, 15%, 20%, 25%, 30%, 35% and 40%. In some cases, a percentage of the length of the distal region 412 compared to the length of the proximal region 425 ranges between any two of the following values: 1%, 5%, 20%, 40%, 50% and 60%.

The diameter (also referred to as profile) of the tether 478 can be sufficiently small to be positioned within the anchor delivery catheter such that the anchor delivery catheter can be maneuvered (e.g., atraumatically) through the patient's blood vessels and/or heart. The diameter of the tether 478 may be limited by size limitations of the associated devices, such as the catheter(s) that the tether 478 will be housed in and/or the prosthetic valve that will travel over the tether 478. In some cases, the distal region 412 and the proximal region 425 can have (e.g., substantially) the same diameter, which may provide a continuous diameter along the length of the tether 478. In some examples, a diameter of at least the distal region 412 of the tether 478 may range between any two of the following values: 0.02 inches, 0.03 inches, 0.04 inches, 0.05 inches, 0.06 inches, 0.07 inches and 0.08 inches. In some examples, an average diameter of the tether 78 may range between any two of the following values: 0.02 inches, 0.03 inches, 0.04 inches, 0.05 inches, 0.06 inches, 0.07 inches and 0.08 inches.

The distal region 412 and the proximal region 425 can have different structural and/or performance characteristics. For example, the distal region 412 and the proximal region 425 can have different flexibility characteristics. In some examples, the distal region 412 is more flexible than the proximal region 425. A more flexible distal region 412 may allow the distal region 412 to sufficiently bend as is traverses the patient's blood vessels, transeptally enter the left atrium and/or within the tight spaces around the native valve. In some examples, the distal region 412 is configured to have a variable flexibility. For example, the distal region 412 can be configured to transition between a flexible state and a stiff state based on a user's input. In some cases, the distal region 412 takes on a pre-determined shape when in the stiff state. Such variable flexibility may allow the distal region 412 to be steerable. This can be useful, for example, for using the tether 478 to adjust a position of the anchor 15 during deployment of the anchor 15 and/or once the anchor 15 has been deployed around the chordae. In some cases, a tether with a steerable distal region 412 can replace a positioning tool (e.g., 206 in FIGS. 2C-2I).

### Flexible Distal Region

As described above, in some examples, the distal region 412 of the tether 478 is more flexible than the proximal region 425 of the tether 478. The distal region 412 have any of a variety of structural characteristics and be constructed using any of a variety of materials. A more flexible distal region 412 may allow the distal region 412 to sufficiently bend as is traverses the patient's blood vessels and/or enters the left atrium transeptally. The relative flexibility of the distal region 412 of the tether 478 may allow the distal region 412 to fit within tight spaces around the native valve. For example, the distal region 412 may be flexible enough to not interfere with the geometry of the anchor guide or other portions of the anchor delivery catheter. In some examples, the distal region 412 has a target minimum bend radius of about 3 mm. The relative stiffness of the proximal region 425 of the tether 478 may allow the proximal region to withstand elongation and/or compression under axial load. Although the proximal region 425 may be relatively stiff compared to the distal region 412, proximal region 425 may still be flexible enough to sufficiently bend to some degree.

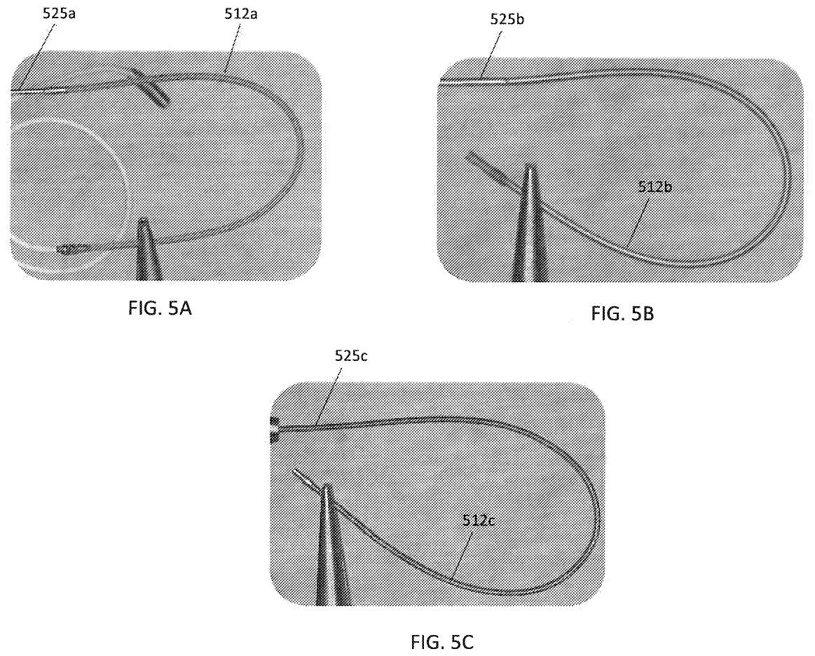

FIGS. 5A-5C illustrate examples of different types of tethers. FIG. 5A shows an exemplary tether having a distal region 412a that includes a cable housing (e.g., stainless steel), which is welded to a proximal region 525a that includes a metal wire (e.g., NiTi). FIG. 5B shows an exemplary tether having a distal region 512b that includes a flexible hypotube (e.g., metal, such as stainless steel), which is welded to a proximal region 525b that includes a stiffer hypotube (e.g., metal, such as stainless steel). FIG. 5D shows an exemplary tether having a distal region 412c that includes a high pics per inch (PPI) braid over a closed pitch coil, which is welded to a proximal region 525c that includes a low PPI braid over a closed pitch coil. As shown in the examples of FIGS. 5A-5C, the distal regions of a tether may be sufficiently flexible to form at least an L-shape or a U-shape bend. Such a degree of flexibility may be useful, for example, in cases where at least a portion of the distal region of the tether loops within the left ventricle and forms a U-shaped bend in an inverted configuration.

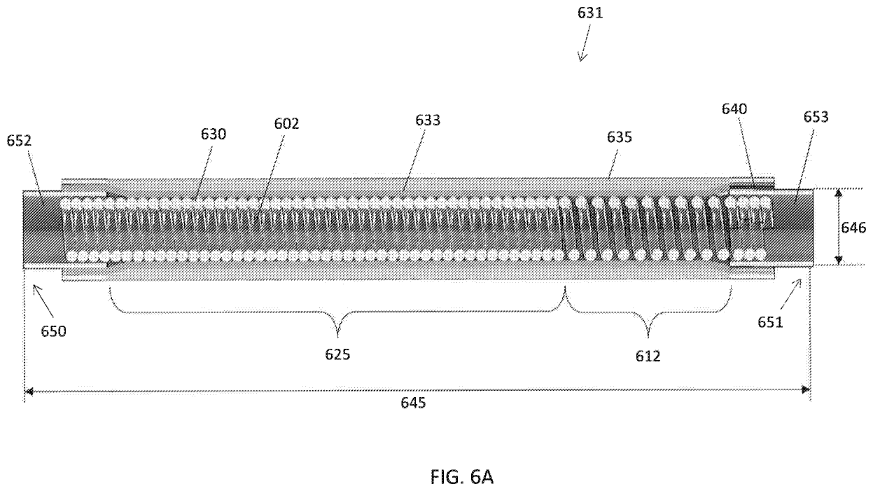

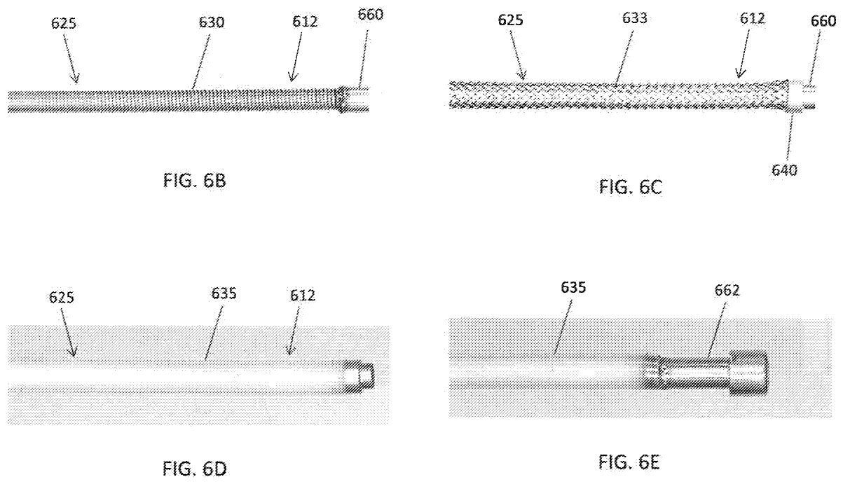

As described herein, in some examples, the tether may include a cable housed within a tubular housing. The tubular housing may have one or more features that provide a distal region and a proximal region with different flexibilities. FIG. 6A shows a section view of an exemplary tubular housing 631 of a tether. The housing 631 can have a tubular shape with an inner lumen 602 that is sized and shaped to house a cable (also referred to as a wire). The tether housing 601 can include a distal region 612 and a proximal region 625, where the distal region 612 has more flexibility (e.g., lateral bendability) than the proximal region 625. The distal region 612 may have a shorter axial length than the proximal region 625. The housing 631 can include a first end 650 and a second end 651. The first end 650 can include a first tubular hub 652, and the second end 651 can include a second tubular hub 653. The second tubular hub 653 may be configured to couple with a connector that is configured to releasably couple to the anchor. The first tubular hub 652 may be configured to couple with a control (also referred to as an actuator) for controlling aspects of the tether. For example, the actuator may control aspects of the connector (e.g., 20, FIGS. 1G and 4A), such as retention and/or release of the anchor.

In some examples, the tubular housing 601 can have a layered structure. For example, the housing 601 can include one or more of the following: a tubular coil 630, a braided tube 633, and a tubular jacket 635. In some cases, the coil 630 is positioned within the braided tube 633, with the jacket 635 positioned around the of the braided tube 633.

In some cases, the tubular coil 630 can have a varied pitch along a length of the coil 630. For example, a distal region 612 of the coil 630 can have a relatively open pitch to provide greater flexibility. A proximal region 625 of the coil 630 can have a relatively closed pitch to provide higher column strength and resistance to kinking.

In some cases, the braided tube 633 can include a varied pitch along a length of the braided tube 633. For example, a distal region 612 of the braided tube 633 can have a relatively high PPI to provide greater flexibility. A proximal region 625 of the braided tube 633 can have a relatively low PPI for anchor tracking.

In some cases, the tubular jacket 635 can be made of a material having a varied durometer along a length of the jacket 635. For example, a distal region 612 of the jacket 635 can have a relatively low durometer to provide greater flexibility. A proximal region 625 of the jacket 635 can have a relatively high durometer for anchor tracking.

In some examples, the second tubular hub 653 may include one or more markers 640 that is visible by imaging techniques. For example, the marker(s) 640 may be radio-opaque markers visible by radiographic techniques such as X-ray, CT scan and/or CAT scan. In some cases, the marker(s) are bands that radially surround the lumen 602 of the housing 631. In some cases, the marker band(s) are secured between the second tubular hub 653 and the braided tube 633.

In some examples, a length 645 of the tubular housing 631 ranges between any two of the following values: 30 inches, 50 inches, 80 inches, 100 inches, 150 inches and 200 inches. In some examples, an outer diameter 646 of the first tubular hub 652 and/or the second tubular hub 653 ranges between any two of the following values: 0.02 inches, 0.03 inches, 0.04 inches, 0.05 inches, 0.06 inches, 0.07 inches and 0.08 inches.

FIGS. 6B-6E show an exemplary tubular housing 631 during various stages of manufacture. FIG. 6B shows an exemplary tubular coil 630 having a distal region 612 and a proximal region 625 with different coil pitches. The distal region 612 may have a more open coil pitch (with larger spaces between adjacent coil windings) relative to the tighter coil pitch proximal region 625. The more open coil pitch can give the distal region 612 a greater flexibility (e.g., lateral bendability) compared to the proximal region 625. The tighter coil pitch of the proximal region 625 can provide a greater column strength and resistance to kinking. A distal end of the tubular coil 630 can include a tubular element 660, which can be part of the distal tubular hub 653 (See, e.g., FIG. 6A).

FIG. 6C shows an exemplary braided tube 633 positioned around the tubular coil 630. The braided tube 633 can have a varied pitch. For example, a distal region 612 of the braided tube 633 can have higher PPI relative to the proximal region 625. The higher PPI can give the distal region 612 a higher flexibility (e.g., lateral bendability) compared to the proximal region 625. The lower PPI of the proximal region 625 can provide a greater column strength and resistance to kinking. In some cases, the distal end of the braided tube 633 can include one or more marker bands 640 that is/are visible by imaging techniques.

FIG. 6D shows an exemplary tubular jacket 635 positioned around the braided tube 633. In some cases, the tubular jacket 635 can be made of one or more polymer materials. The polymer materials have a varied durometer along a length of the jacket 635. For example, a distal region 612 of the jacket 635 can have a relatively low durometer to provide greater flexibility compared to the proximal region 625.

FIG. 6E shows the tether 631 after a distal element 662 is attached. The distal element may include a connector (e.g., 20, FIGS. 1G and 4A) that is configured to releasably connect to the anchor. The proximal end of the tether 631 may be coupled to an actuator for actuating one or more aspects of the tether and/or the connector.

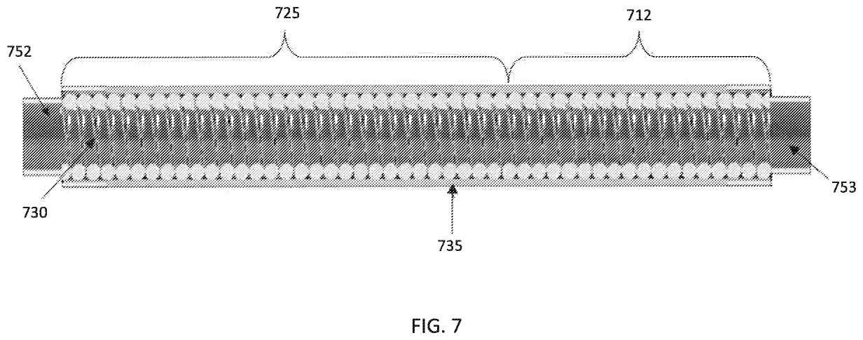

FIG. 7 illustrates another example of tether housing 731 having similar features as the housing 631 in FIG. 6A except that the tubular coil 730 does not have a varied pitch and the housing 731 does not include a braided tube (e.g., 633). The jacket 735 can be made of polymer material(s) have a varied durometer along a length of the jacket 735 such that the distal region 712 is more flexible than the proximal region 725 upon compression. The tubular coil 730 can have a tight pitch, thereby providing performance with respect to resistance to elongation/compression.

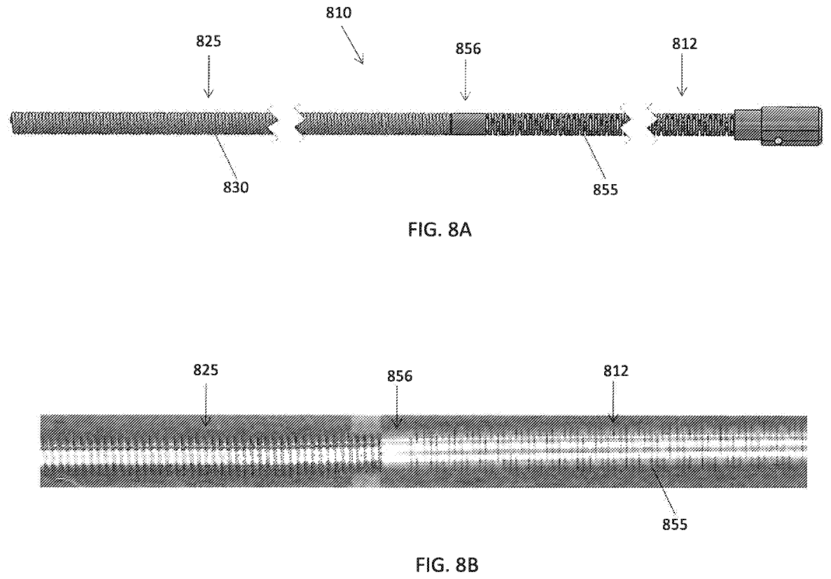

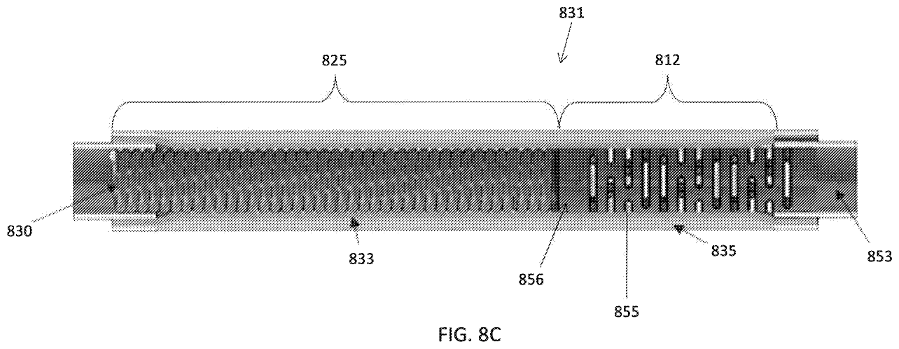

FIGS. 8A-8C illustrate another exemplary tether housing 831. The housing 831 includes a bendable component 810 having similar features as the tubular coil 630 in FIG. 6A except that the distal region 812 includes a slitted tube 855 instead of a tubular coil. As used herein, an interruption or gap in a wall of a tube may be referred to as a “slit,” “window,” “gap,” “fenestration,” or “aperture.” As shown in FIGS. 8A and 8B, the proximal region 812 can include a stacked tubular coil 830 (similar tubular coil 630 of FIG. 6A) that is coupled to the slitted tube 855 at a junction region 856 (e.g., by welding). The slitted tube includes a number of transverse slits 855 that are configured to increase the flexibility and lateral bendability of the distal region 812. The slits 855 may be formed, for example, by laser cutting. In some examples, the slits 855 are arranged in an interrupted spiral pattern. FIG. 8C shows the tubular coil 830 and the slitted tube encased within a braided tube 833 and a jacket 835. In some cases, a distal region 812 of the braided tube 833 can have a relatively high PPI compared to the proximal region 825 to provide greater flexibility. In some cases, a distal region 812 of the jacket 835 can have a relatively low durometer compared to the proximal region 825 to provide greater flexibility.

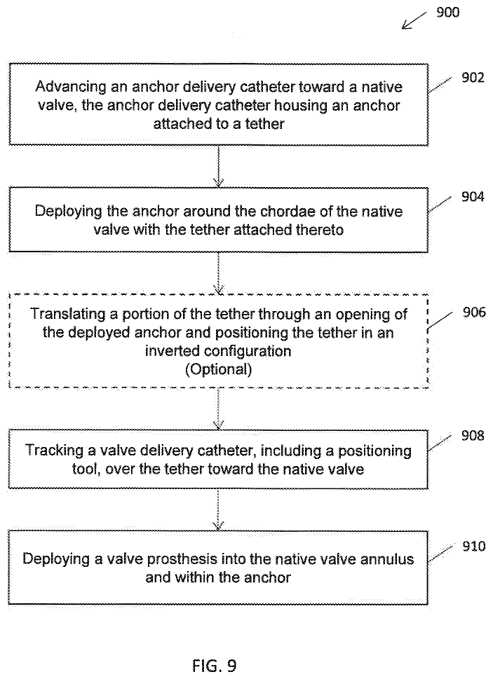

FIG. 9 is a flowchart 900 illustrating an example method of using a tether having a flexible distal region. At exemplary operation 902, an anchor delivery catheter (e.g., as part of anchor delivery device or system) is advanced through a patient's blood vessel, into the patient's heart and toward a native heart valve (e.g., mitral valve). The anchor delivery catheter can house an anchor and tether therein, where the anchor is attached to the tether within the anchor delivery catheter. The tether can include a distal region and a proximal region, where the distal region is more flexible than the proximal region. During advancement of the tether, the flexibility of the distal region of the tether may allow the distal region to traverse the patient's blood vessels and transeptally enter the left atrium while the tether is within the anchor delivery catheter.

At exemplary operation 904, the anchor is deployed around the chordae and/or leaflets of a native valve. The anchor can have a spiral shape that wraps around and engages with at least some of the chordae of the native valve. In some cases, the anchor is deployed from an anchor guide (e.g., as part of the anchor delivery device or system). A portion of the tether can extend from the anchor delivery catheter and maintain a connection to the anchor. A flexible distal region may allow the anchor to move more freely during deployment, thereby reducing the risk of de-encircling the anchor. A more flexible distal region may also allow the distal region to bend to a smaller bend radius, which can make the tether more maneuverable within tight spaces. The flexibility in the tether may be designed to be inefficient in transferring loads to the anchor to reduce the risk of disturbing anchor position. That is, translation of compression axial loads from the tether to the anchor may be muted, thereby assisting in preserving anchor placement, even under tether manipulation. In addition, if an anchor guide is used, a flexible distal region may allow the tether and anchor to be pushed out of the anchor guide during deployment, and improve the encircling of the anchor around the chordae. For example, the tether may be required to form at least an L-shaped bend.

At exemplary operation 906, a portion of the tether may optionally be translated through a central opening of the deployed anchor and the tether may be positioned in an inverted configuration (See, e.g., FIGS. 3A-3H). At least a portion of the distal region may be positioned in an inverted configuration inferior to the deployed anchor (e.g., within the left ventricle). The distal region of the tether may be sufficiently flexible to bend and pass through the deployed anchor. In addition, the distal region of the tether may be sufficiently flexible to take on an inverted configuration (e.g., including a U-shaped bend).

At exemplary operation 908, the valve delivery catheter may be tracked over the tether toward the native valve. The tether, including the distal region and the proximal region, can have sufficient tensile strength to the support the valve delivery catheter as it traverses along the tether. In some examples, a positioning tool (e.g., 206, FIGS. 2C-2I) is tracked over the tether toward the connection between the tether and the anchor. The positioning tool can be a hollow member that is capable of mating with the anchor (or an attachment attached to the anchor). In some examples, the positioning tool can be used to adjust a position of the anchor to achieve axial alignment between the anchor and the valve delivery catheter for proper alignment with the prosthetic valve. In cases where the tether is in an inverted configuration, the positioning tool may also take on an inverted configuration. For example, the positioning tool can be configured to follow along the tether through the central opening of the anchor and into the ventricle, and take on a U-shaped bend to engage with the anchor.

At exemplary operation 910, the valve prosthesis is deployed from the valve delivery catheter system and into the native valve annulus and within the anchor. Once the valve prosthesis is fully deployed, the tether can be disconnected from the anchor and removed from the heart and the patient's body, along with other components of the valve delivery catheter system, leaving the prosthetic valve and anchor secured in place within the heart.

### Variably Flexible Tether

In some examples, at least a portion of the tether is variably flexible, where the tether can transition between a flexible state and a stiff state. The tether may also be configured to bend as it is stiffened. The tether may be configured to actively bend along one or more regions of the tether, as controlled by a user. Returning to FIG. 4D, for example, in some cases, the distal region 412 of the tether 478 can be variably flexible. When in the flexible state, the distal region 412 can be flexible enough to sufficiently bend as is traverses the patient's blood vessels, transeptally enter the left atrium, and/or pass through the anchor to assume an inverted configuration. In some cases, when in the flexible state, the distal region 412 can be more flexible than the proximal region 425. In some cases, when in the stiff state, the distal region 412 can be less flexible than the proximal region 425. When in the stiff state, the distal region 412 may be sufficiently stiff to control movement of the anchor 15 during one or more procedures during anchor deployment and/or prosthetic valve deployment. For example, when in the stiff state, the distal region 412 may be stiff enough to control the axial height of the anchor before and/or during deployment of the valve prosthesis. In some cases, when in the stiff state, the distal region 412 can be stiffer than the proximal region 425. In some cases, when in the stiff state, the distal region 412 can be less stiff than the proximal region 425. In some cases, the position of the anchor 15 can be adjusted by transitioning the anchor between the flexible and stiff states.

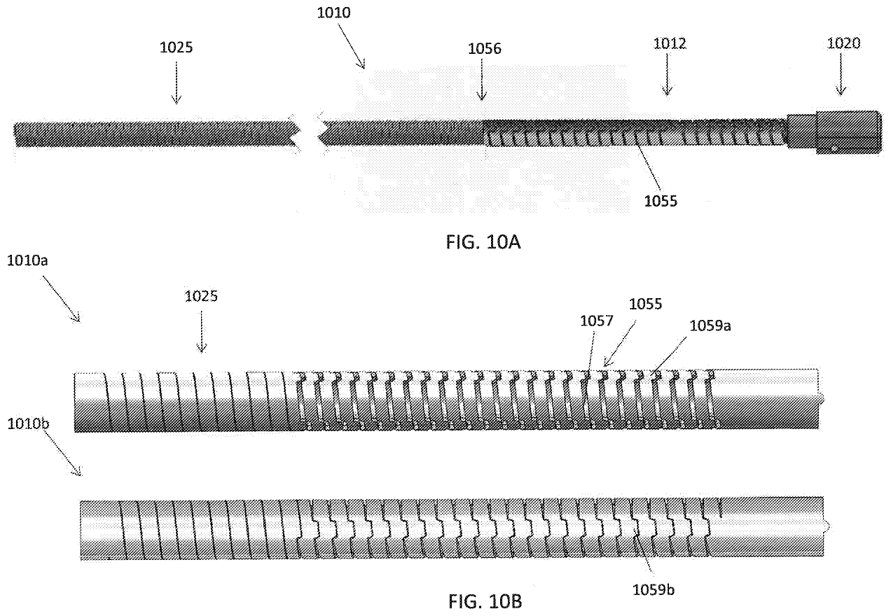

FIG. 10A illustrates an exemplary preferential bendable tubular component 1010 of a tether housing that is configured to provide a variable flexibility. A proximal region 1025 can include a stacked tubular coil 730 (e.g., similar tubular coil 630 of FIG. 6A or tubular coil 730 of FIG. 7A). The preferential bendable component 1010 may be covered with one or more outer sheaths or jackets (not shown). The proximal region 1025 can be coupled to a distal region 1012 at a junction region 1056 (e.g., by welding). In this case, the proximal region 1025 includes a stacked coil, and the distal region 1012 includes a laser-cut hypotube. Both the proximal region 1025 and the distal region 1012 may be comprise one or more metals, e.g., stainless steel, elgiloy and/or CoCr. The distal portion 1012 may be coupled to a connector 1020 that is configured to releasably connect the tether to an anchor. The distal region 1012 can include a number of transverse slits 1055 that are configured to cause the distal region 1012 to bend to a pre-determined shape when an axially compressive force is placed on the component 1010. For example, the distal region 1012 can be configured to form one or more bends (e.g., L-shape and/or U-shape bends). The slits 1055 may be circumferentially oriented so that that section activates to a complex curvature. The pattern of slits 1055 may twist around the tube to achieve complex curvature.

FIG. 10B shows a closeup view of a variation of component 1010 in top (e.g., 1010a) and bottom (e.g., 1010b) aspects, having a flexible section 1025 and a pattern of the slits 1055 having openings 1057. In some embodiments, an opening of a slit has a relatively uniform (axial) width along its (circumferential) length. In some embodiments, an opening of a slit has a variable width along its length. A variable opening can comprise a taper or a wedge. Each of the slits 1055 can form an opening 1057 that closes upon axial compression, thereby causing the component 1010 to preferentially bend the component 1010 in a direction that closes the openings 1057. The bending can also cause the component 1010 to stiffen as the openings 1057 close. In some embodiments, at least one slit 1055 includes features 1059a and/or 1059b that are configured to prevent rotation (torsion) of the component 1010. The flexible section 1025 can include a tubular coil and/or a spiral cut portion.

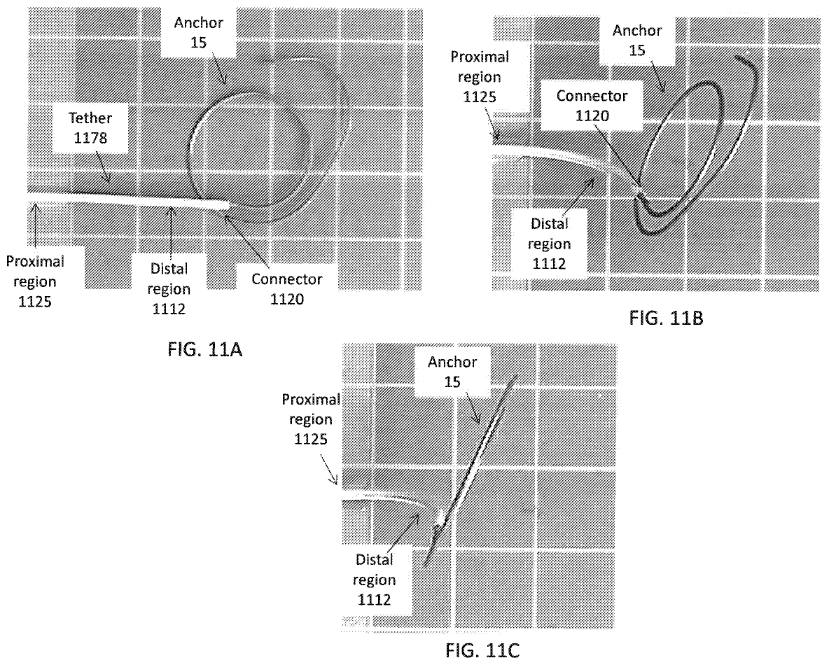

FIGS. 11A-11C illustrate an example tether 1178 having variable flexibility. FIG. 11A shows the tether 1178 attached to an anchor 15, where the distal region 1112 and proximal region 1125 of the tether 1178 are in straight configurations. In FIG. 11A, the distal region 1112 can be in a flexible state so that it can sufficiently bend during entry into the heart (e.g., while within the anchor delivery catheter) and/or maneuvering within the heart. In some cases, the tether 1178 can be sufficiently flexible to be positioned in an inverted configuration when in the flexible state. The proximal region 1125 can be sufficiently stiff to resist axial elongation/compression as the tether 1178 traverses through the patient and/or as the tether 1178 is pushed/pulled through the anchor delivery catheter and/or the valve delivery catheter. In some cases, the distal region 1112 may be able to bend to a bend radius ranging between 3 mm and 5 mm when in a flexible state.

FIG. 11B shows the distal region 1112 in a bent state caused by placing axial compression on the distal region 1112. This can be accomplished by pulling (or in some cases pushing) the inner cable (e.g., 480, FIGS. 4B and 4C) relative to the tether housing (e.g., 401, FIGS. 4B and 4C). For example, the connector 1120 can be configured to prevent axial movement (e.g., distal and/or proximal) movement of the inner cable relative to the tether housing when the connector 1120 is in a locked state. Thus, pulling (or in some cases pushing) the inner cable can place axial compression on the inner cable, thereby causing the tubular tether housing to bend. The distal region 1112 can also become stiffer as it bends. FIG. 11C shows the distal region 1112 after further axial compressive force is applied to the distal region 1112, causing the distal region 1112 to bend and stiffen further. In this case, the distal region 1112 is configured to take on a final shape having an L-shaped bend such that the anchor 15 is in an orthogonal orientation compared to its initial orientation (e.g., FIG. 11A). The tether 1178 can be configured to achieve a maximum stiffness once the tether 1178 takes on a final (e.g., predetermined) shape (e.g., when at least a portion of the openings 1057 of slits 1055 fully close).

In some cases, an actuator (e.g., actuator 421) can be configured to allow a user to control a degree of bending/stiffening of the distal region 1112. For example, the actuator may be configured to allow the user to stiffen the distal region 1112 from a relatively flexible state (e.g., FIG. 11A) to a partially bent/stiffened state (e.g., FIG. 11B), and bend/stiffen further to a final bent/stiffened state (e.g., FIG. 11C). This can allow the user to choose an amount of bending/stiffness of the distal region 1112 as needed to properly adjust the position of the anchor within the heart. The actuator can be configured to allow the user to straighten/unstiffen the distal region 1012 through the various states of bending/stiffness. In some cases, the actuator is configured to allow a user to lock the shape/stiffness of the distal region 1112 in one or more degrees of bending/stiffness.

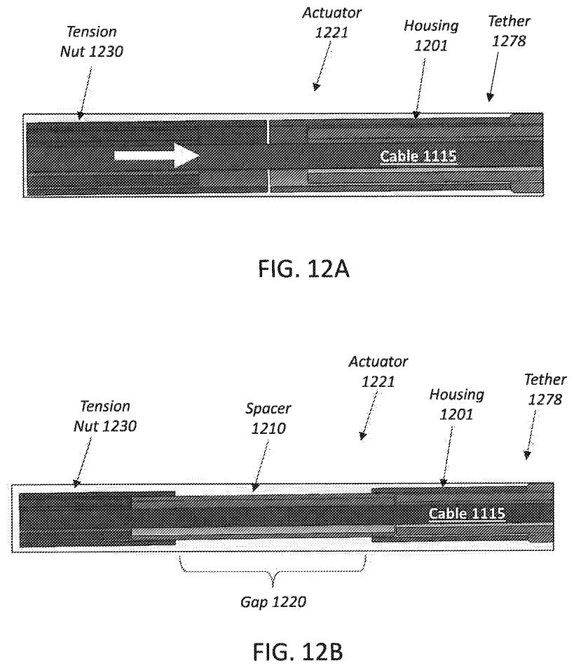

FIGS. 12A and 12B show an example actuator 1221 for retaining the anchor. FIG. 12A shows the actuator 1221 when the tether is disengaged from the anchor, and FIG. 12B shows the actuator 1221 when the tether is engaged and locked with the anchor. The actuator 1221 can be at a proximal end of the tether 1278, e.g., as part of a handle. An internal cable 1215 is coupled to the anchor 15 within a tubular housing 1201 of the tether 1278. The actuator 1221 includes a removable spacer 1210 that fits within a gap 1220 between the housing 1201 of the tether 1278 and a tension nut 1230. The cable 1215 can be held under tension when the spacer 1210 is positioned within the gap 1215. The actuator 1221 is configured to control the engagement of the anchor from the tether 1278. For example, the actuator 1221 may be configured to cause a connector (e.g., connector 20, FIGS. 1G, 4A-4D) to engage with the anchor and couple the anchor to the tether 1278 when the spacer 1210 is within the gap 1220. FIG. 12A shows the actuator 1221 after the spacer 1210 has been removed from the gap 1220 between the tubular housing 1201 and the tension nut 1230. Once the spacer 1210 is removed, the gap 1220 can narrow as the tension nut 1230 and the cable 1215 move distally.

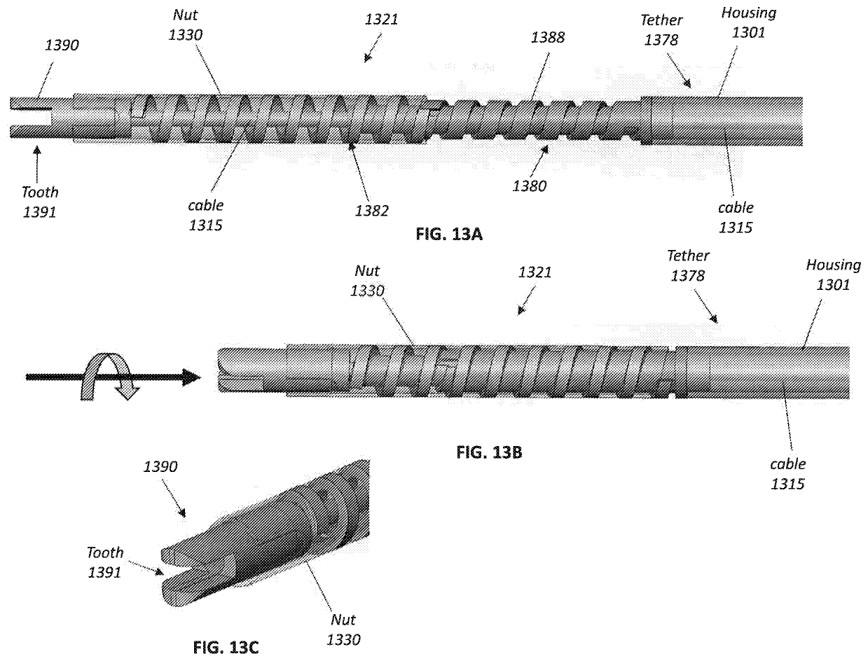

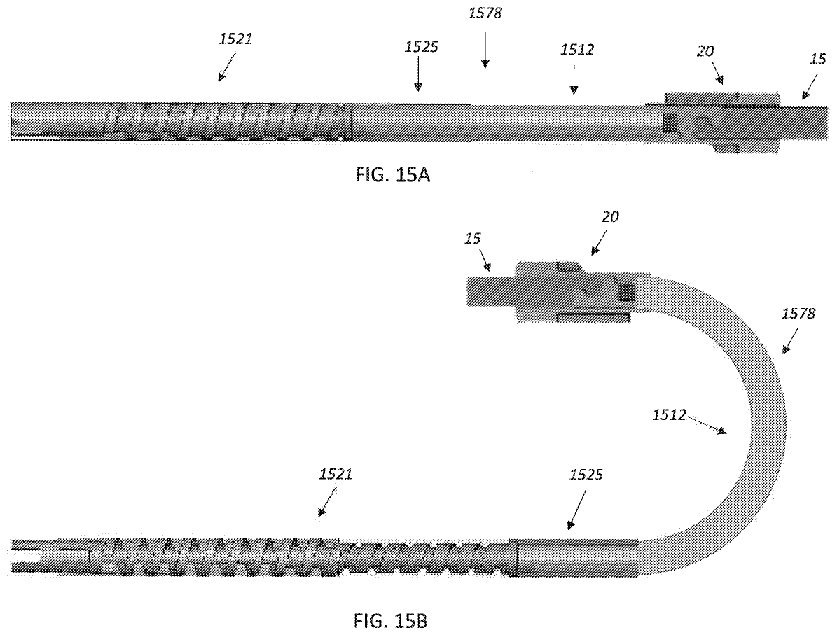

FIGS. 13A-13C illustrate another exemplary actuator 1321, in this case, having a lead screw configuration. The proximal end 1388 of the housing 1301 of the tether 1378 has a threaded outer surface 1380 that is configured to engage with a threaded opening 1382 of a proximal nut 1330. FIG. 13A shows the actuator assembly 1321 applying tension to the cable 1315 such that the distal region of the tether 1378 can take on a stiff and bent state. Rotation of the tooth 1391 and cable 1315 relative to the nut 1330 and the housing 1301 can cause the tooth 1391 and the cable 1315 to translate distally relative to the housing 1301, as shown in FIG. 13B, thereby releasing some tension in the cable 1315 and causing the distal region of the tether 1378 to relax to a flexible state. FIG. 13C illustrates a proximal end 1390 of the actuator 1321 showing the tooth 1391, which may include a slot for accepting a piece that facilitates rotation of the tooth 1391. In some examples, the actuator 1321 is also configured to control the engagement of the anchor from the tether 1378. For example, the actuator 1321 may be configured to cause a connector (e.g., connector 20, FIGS. 1G, 4A-4D) to engage with the anchor and couple the anchor to the tether 1378 when tension is placed on the cable 1315. For example, the locked state of the anchor may be midway through the leadscrew travel (e.g., a released state similar to FIG. 15A and an activated state similar to FIG. 15B).

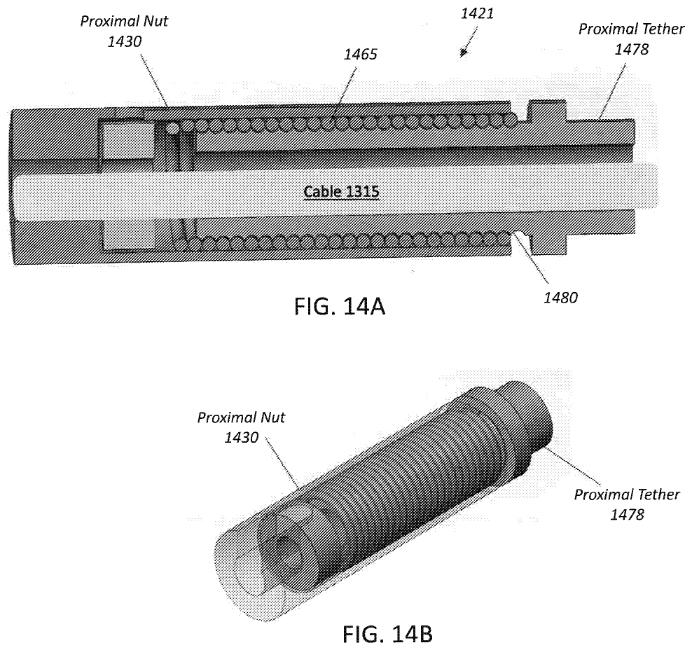

FIGS. 14A and 14B illustrate another variation of an actuator assembly 1421 having a similar lead screw configuration as the actuator assembly 1321 except that the internal threads within the proximal nut 1430 are formed by a coil 1465. In some cases, the external threads at the outer surface 1480 of the proximal end of the tether 1478 can be formed from a ball end mill. This construction may have some manufacturability advantages over the actuator assembly 1321.

In either of the screw-based actuator assemblies 1321 and 1421, the pitch of the threads may vary depending on design requirements. For example, the pitch may be fine enough to allow the user to adequately control the bending and stiffening of the distal region of the tether but not too fine such that the user is encumbered by having to make too many turns of the actuator 1321/1421. FIGS. 15A and 15B illustrate an exemplary tether system, which includes a tether 1578 having a proximal end attached to an actuator 1521 and a distal end removably attached to an anchor 15 via a connector 20. In FIG. 15A, the actuator 1521 maintains the tether 1578 in a nominally flexible state where at least a proximal region 1512 of the tether 1578 is in a flexible state. The actuator 1521 may also cause the connector to engage with and retain the anchor 15 thereto. FIG. 15B shows the actuator 1521 after it has been manipulated (e.g., rotated) to place tension on the inner cable of the tether 1578 proximally, thereby causing at least the distal region 1512 of the tether 1578 to bend and stiffen. In this case, the proximal region 1512 of the tether 1578 forms a U-shaped bend (e.g., inverted configuration), which can provide access to the anchor 15 from an inferior position (e.g., within the left ventricle). The stiffness of the distal region 1512 and the proximal region 1525 of the tether 1578 can allow the user to control a position of the anchor 15 (e.g., once deployed around the chordae) via movement of the anchor 15 as the tether 1578 is transitions from the flexible/straight state (FIG. 15A) to the bent/stiff state (FIG. 15B). In some cases, the actuator 1521 is also configured to control the release of the anchor 15 from the tether 1578.

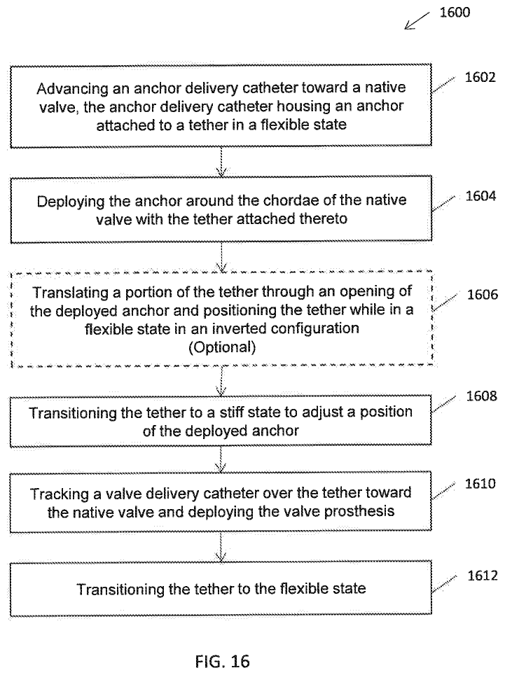

Advantageously, the variably flexible tethers described herein can be used during delivery of an anchor and/or valve without requiring the use of a positioning tool. FIG. 16 is a flowchart 1600 illustrating an example method of using a variably flexible tether in place of a positioning tool (e.g., without the positioning tool 206 in FIGS. 2C-2I). At exemplary operation 1602, an anchor delivery catheter (e.g., as part of anchor delivery device or system) is advanced through a patient's blood vessel, into the patient's heart and toward a native heart valve (e.g., mitral valve). The anchor delivery catheter can house an anchor and tether therein, where the anchor is attached to the tether within the anchor delivery catheter. At least a portion of the tether (e.g., a distal region) can be configured to transition between a flexible state and a stiff state (variably flexible). The tether can be in a flexible state as the anchor delivery catheter is delivered to the heart.

At exemplary operation 1604, the anchor is deployed around the chordae of a native valve. The anchor can have a spiral shape that wraps around and engages with at least some of the chordae of the native valve. In some cases, the anchor is deployed from an anchor guide (e.g., as part of the anchor delivery device or system). A portion of the tether can extend from the anchor delivery catheter and remain connected to the anchor. At least a portion of the variably flexible region (e.g., distal region) of the tether can extend from the anchor delivery catheter and remain connected to the anchor. The variably flexible region (e.g., distal region) of the tether can remain in the flexible state to allow the anchor to freely deploy around the chordae without causing the anchor to de-encircle. In some cases, the variably flexible region may be able to bend to a bend radius ranging between 3 mm and 5 mm when in the flexible state.

At exemplary operation 1606, a portion of the tether may optionally be translated through a central opening of the deployed anchor while the tether is in a flexible state. This operation may be used in cases where the tether is positioned in an inverted configuration (e.g., FIG. 3H). At exemplary operation 1608, the tether can be transitioned to a stiff state to adjust a position of the deployed anchor. In some cases, a position of the anchor is adjusted to achieve axial alignment between the anchor and the valve delivery catheter for proper alignment with the prosthetic valve. In some cases, the tether takes on a pre-determined shape when in a (e.g., fully) stiff state. In some cases, the pre-determined shape includes an L-shaped bend. In cases where the tether is in an inverted configuration, stiffening the tether may adjust the anchor from an inferior position relative to the anchor (e.g., sub-annular to the native valve). In some cases, the pre-determined shape in the inverted configuration includes a U-shaped bend.

At exemplary operation 1610, a valve delivery catheter may be tracked over the tether toward the native valve. Once the valve delivery catheter is properly aligned with the native valve annulus, the valve prosthesis can be deployed (e.g., expanded) into the native valve annulus and the anchor. At exemplary operation 1612, the tether can be transitioned back to the flexible state. This can allow the tether to be flexible enough to assume a substantially straight configuration for retracting back into the valve delivery catheter. The tether can also be disconnected from the anchor (e.g., via connector 20). The tether and other portions of the valve delivery catheter can be retracted and removed from the patient body, leaving the valve prosthesis and anchor secured in place within the heart.

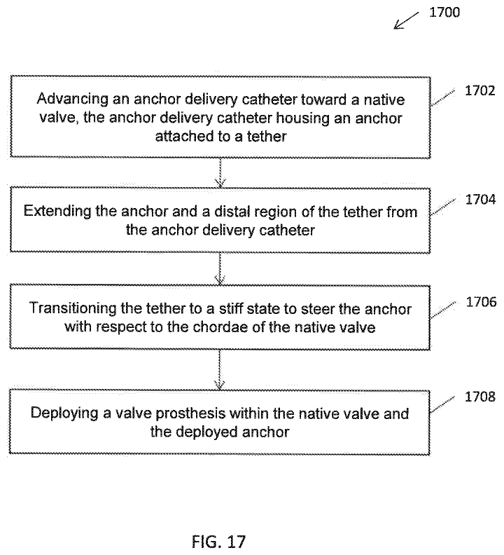

Advantageously, the variably flexible tethers described herein can be used during delivery of an anchor and/or valve without requiring the use of an anchor control catheter and/or anchor guide. FIG. 17 is a flowchart 1700 illustrating an example method of using a variably flexible tether in place of an anchor guide (e.g., 153 in FIGS. 1C-IF or 112 in FIG. 3A). At exemplary operation 1702, an anchor delivery catheter (e.g., as part of anchor delivery device or system) is advanced through a patient's blood vessel, into the patient's heart and toward a native heart valve (e.g., mitral valve). The anchor delivery catheter can house an anchor and tether therein, where the anchor is attached to the tether within the anchor delivery catheter. The tether can include a variable distal flexible region that is able to transition between a flexible and stiff state. During advancement of the tether, the distal region of the tether may be in a flexible state to allow the distal region to traverse the patient's blood vessels and transeptally enter the left atrium while the tether is within the anchor delivery catheter.

At exemplary operation 1704, the anchor and at least a portion of the distal region of the tether is extended from the distal end of the anchor delivery catheter (also referred to herein as an outer sheath). At exemplary operation 1706, the tether (e.g., distal region) is transitioned to a stiff state to steer the anchor with respect to the chordae and/or leaflets of the native valve. The stiffness of the tether (e.g., distal region) can allow the tether to control the position and orientation of the anchor so that a user can steer the anchor. In some cases, the distal region of the tether takes on a curved (e.g., pre-determined) shape as it is transitioned from the flexible state to the stiff state. The curvature of the distal region in the stiff state may advantageously orient the anchor with respect to the chordae and/or leaflets of the native valve.

At exemplary operation 1708, after the anchor is deployed in proper position, the valve delivery catheter may be used to deploy the prosthetic valve into the native valve annulus and the anchor, as described herein. In some cases, the valve delivery catheter is deployed over the tether while in a stiff state. In other cases, the valve delivery catheter is deployed over the tether while in a flexible state.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A complete listing of the claims follows:

1. A system for treating a diseased native valve of a heart, the system comprising:
an anchor having a spiral shape that is configured to engage with chordae tendineae and/or leaflets of the diseased native valve and to provide a securing force around a valve prosthesis; and
a tether configured to provide access to the anchor when the anchor is deployed around the chordae tendineae and/or leaflets of the native valve, the tether having a distal region and a proximal region, the distal region having a distal end connected to the anchor, wherein the distal region and the proximal region have different bending stiffnesses.

2. The system of claim 1, wherein the tether has a sufficiently high tensile strength to support an anchor delivery catheter and a valve delivery catheter tracked thereover.

3. The system of claim 1, wherein the distal region has a sufficiently high tensile strength to allow a user to manipulate a position of the anchor when the anchor is deployed around the chordae of the native valve.

4. The system of claim 1, wherein the proximal region has a sufficiently high compressive strength to resist axial compression when an axial compressive force is applied to the tether, and a sufficiently high tensile strength to resist axial elongation when an axial tensile force is applied to the tether.

5. The system of claim 1, wherein the distal end of the tether is releasably connected to the anchor.

6. The system of claim 1, wherein the distal region has a lower bending stiffness than the proximal region.

7. The system of claim 1, wherein the tether includes an inner cable housed within a tubular housing.

8. The system of claim 7, wherein the cable is translatable within the tubular housing.

9. The system of claim 8, wherein the tubular housing includes one or more of: a tubular coil, a braided tube, and a tubular jacket.

10. The system of claim 9, wherein the tubular coil at the distal region of the tether has a larger pitch than the tubular coil at the proximal region of the tether.

11. The system of claim 9, wherein the braided tube at the distal region of the tether has a higher pics per inch (PPI) braid than the braided tube at the proximal region of the tether.

12. The system of claim 9, wherein the tubular jacket at the distal region of the tether is made of a low durometer material than the tubular jacket at the proximal region of the tether.

13. The system of claim 8, wherein the tubular housing includes one or more radio-opaque markers.

14. The system of claim 8, wherein the tubular housing at the proximal region includes a stacked tubular coil, and the tubular housing at the distal region includes a slitted tube.

15. The system of claim 1, wherein the distal region is configured to transition between a flexible state and a stiff state, wherein the distal region has a pre-determined shape when in the stiff state.

16. The system of claim 15, wherein the pre-determined shape includes an L-shaped bend.

17. The system of claim 15, wherein the pre-determined shape includes a U-shaped bend.

18. The system of claim 1, wherein the distal region has a shorter length than the proximal region.

19. The system of claim 18, wherein a length of the distal region ranges between 5% and 20% of a length of the proximal region.

20. (canceled)

21. The system of claim 1, wherein the distal region has an axial length that is at least double an axial length of the valve prosthesis.

22.-64. (canceled)
